(19) United States
(12) Patent Application Publication
(10) Pub. No.: US 2015/0245903 A1
(43) Pub. Date: Sep. 3, 2015

(54) TISSUE EXPANDER

(71) Applicant: Hilton Becker, Boca Raton, FL (US)

(72) Inventor: Hilton Becker, Boca Raton, FL (US)

(21) Appl. No.: 14/712,773

(22) Filed: May 14, 2015

(51) Int. Cl. A61F 2/12; A61M 29/02

(52) U.S. Cl. CPC A61F 2/12; A61M 29/02

(57) ABSTRACT

A tissue expander comprising: an outer shell; a first open obloid shell disposed in the outer shell, the first open obloid shell comprising: a first annular face; a first outward face disposed opposing to the first annular face; and a first annular sidewall interposed between the first annular face and the first outward face to connect the first outward face to the first annular face; and a second open obloid shell disposed in the outer shell, the second open obloid shell comprising: a second annular face; a second outward face disposed opposing to the second annular face; and a second annular sidewall interposed between the second annular face and the second outward face to connect the second outward face to the second annular face, wherein the first open obloid shell and the second open obloid shell are stackingly arranged in the outer shell such that the first annular face opposes the second annular face.

### BRIEF DESCRIPTION

Disclosed is a tissue expander comprising: an outer shell; a first open obloid shell disposed in the outer shell, the first open obloid shell comprising: a first annular face; a first outward face disposed opposing to the first annular face; and a first annular sidewall interposed between the first annular face and the first outward face to connect the first outward face to the first annular face; and a second open obloid shell disposed in the outer shell, the second open obloid shell comprising: a second annular face; a second outward face disposed opposing to the second annular face; and a second annular sidewall interposed between the second annular face and the second outward face to connect the second outward face to the second annular face, wherein the first open obloid shell and the second open obloid shell are stackingly arranged in the outer shell such that the first annular face opposes the second annular face.

### BRIEF DESCRIPTION OF THE DRAWINGS

The following descriptions should not be considered limiting in any way. With reference to the accompanying drawings, like elements are numbered alike.

FIG. 1A shows an embodiment of a tissue expander;

FIG. 1B shows an embodiment of a tissue expander;

FIG. 2 shows the tissue expander shown in FIG. 1A in a compressed state;

FIG. 3 shows an embodiment of a tissue expander;

FIG. 4 shows an embodiment of a tissue expander;

FIG. 5 shows an embodiment of a tissue expander;

FIG. 6 shows an embodiment of a tissue expander;

FIGS. 7A, 7B, 7C, and 7D show construction of a tissue expander;

FIGS. 8A, 8B, 8C, 8D, and 8E show construction of the tissue expander;

FIGS. 9A and 9B show an outer shell and an open obloid shell;

FIGS. 10A, 10B, and 10C show construction of the tissue expander;

FIGS. 11A and 11B show construction of the tissue expander;

FIG. 12A shows a tissue expander in a vertical position without folding of the tissue expander; and

FIG. 12B shows an outer shell in a vertical position that folded absence of an open obloid sphere.

### DETAILED DESCRIPTION

A detailed description of one or more embodiments is presented herein by way of exemplification and not limitation.

A tissue expander described herein provides an implantable tissue expander having an internal structural support based on viscoelastic compression properties of silicone bodies such as open obloid shells, which can be a same construction as the outer shell. When the tissue expander is empty, the open obloid shells are compressed and provide a flat expander with internal support that mechanically resists folding or rippling of an outer container in which the open obloid shell is disposed. In response to filling the tissue expander with a fluid such as saline, an internal volume of the open obloid shells expands. As a result, support is provided to the filled tissue expander to maintain a softness and elasticity of the tissue expander effective to simulate healthy breast tissue. Moreover, the open obloid shells do not affect expansion of the outer shell because the outer shell is not structurally connected to the open obloid shell therein. Additionally, the open obloid shell provides internal structural support to the tissue expander such that the outer shell exhibits less rippling or scalloping than an outer shell that does have an open obloid shell.

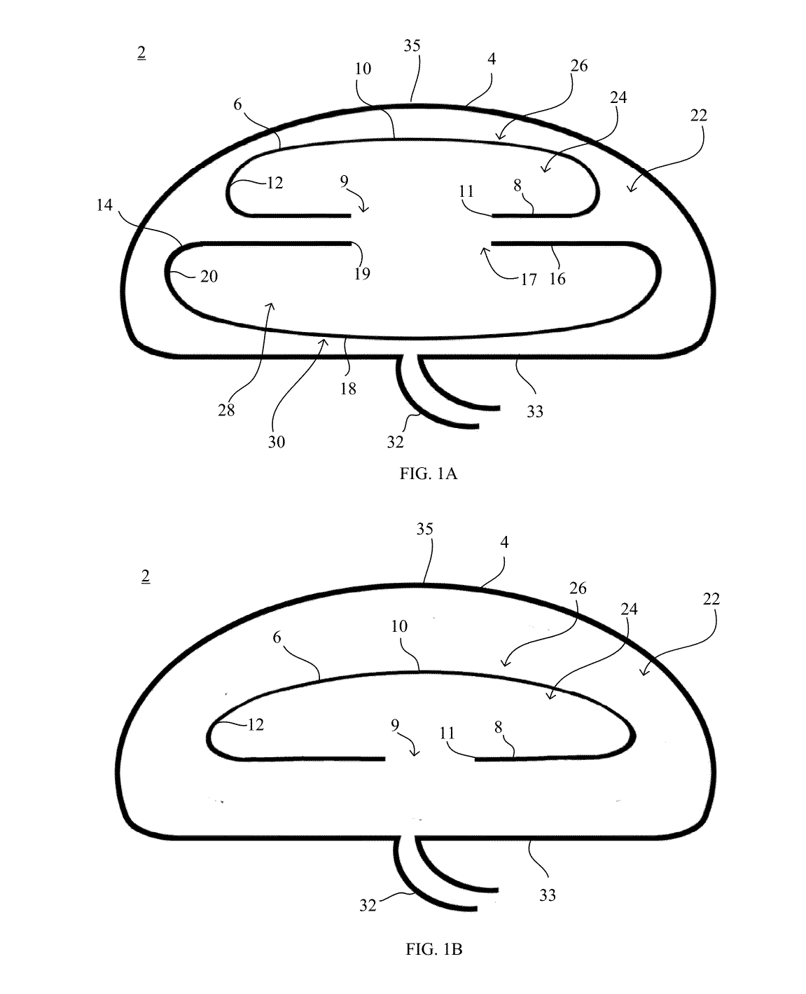

In an embodiment, as shown in FIG. 1A, tissue expander 2 includes outer shell 4 in which first open obloid shell 6 is disposed. First open obloid shell 6 includes first annular face 8, first opening 9 disposed in first annular face 8 and bounded by first rim 11, first outward face 10 disposed opposing first annular face 8, and first annular sidewall 12 interposed between first annular face 8 and first outward face 10 to connect first outward face 10 to first annular face 8. Tissue expander 2 also includes second open obloid shell 14 disposed in outer shell 4, wherein second open obloid shell 14 includes second annular face 16, second opening 19 disposed in second annular face 16 and bounded by second rim 17, second outward face 18 disposed opposing second annular face 16 and second annular sidewall 20 interposed between second annular face 16 and second outward face 18 to connect second outward face 18 to second annular face 16, wherein first open obloid shell 6 and second open obloid shell 14 are stackingly arranged in outer shell 4 such that first annular face 8 opposes second annular face 16. Additionally, outer shell 4 includes interior 22 to contain first open obloid shell 6 and second open obloid shell 14. First open obloid shell 6 also includes interior 24 and exterior 26. It is contemplated that the exterior 26 is coextensive with interior 22. Second open obloid shell 14 includes interior 28 and exterior 30, which is coextensive with interior 22. Filling tube 32 is optionally included in tissue expander 2 to be disposed on posterior surface 33 of tissue expander 2. Filling tube 32 IS disposed on outer shell 4 and includes an inner flow path that is in fluid communication with interior 22 of outer shell 4. Filling tube 32 can also be in fluid communication with first open obloid shell 6 and second open obloid shell 14.

Tissue expander 2 further includes anterior surface 35. In this arrangement, first open obloid shell 6 in combination with second open obloid shell 14 provide a springy, shape-restoring force to outer shell 4 to prevent bending of tissue expander 2. It is contemplated that tissue expander 2 shown in FIG. 1A can be filled with a fluid to selectively expand tissue expander 2 to a selected volumetric size. The fluid can be any fluid compatible with outer shell 4 or human tissue and can include fluid used in FDA-approved medical devices that can be surgically disposed in a patient. Exemplary fluids include water, saline, silicone gel, alcohol, and the like.

In an embodiment, as shown in FIG. 1A, tissue expander 2 can include one open obloid shell 6.

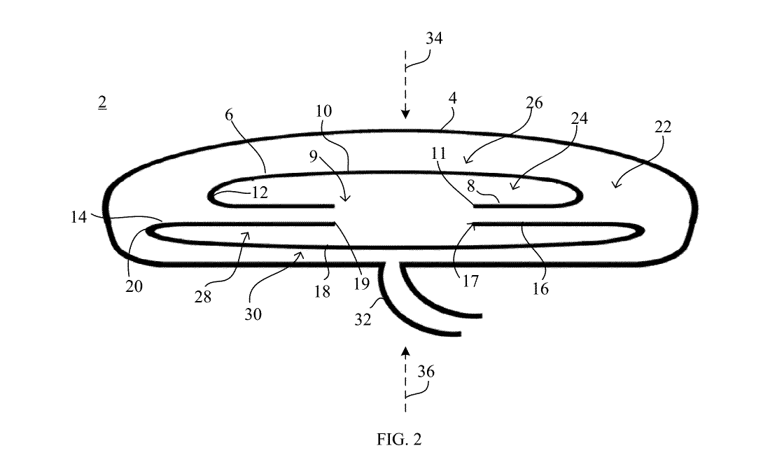

In response to compression, e.g., due to evacuation of fluid from interior 22 of outer shell 4, tissue expander 2 collapses in a direction indicated by arrow 34 and arrow 36 is shown in FIG. 2. Accordingly, outer shell 4 does not fold along the direction orthogonal to wear at an angle with arrow 34 or arrow 36. Further, first open obloid shell 6 and second open obloid shell 14 mechanically resist compression along a direction represented by arrow 34 and arrow 36 and even more strongly resist compression along any other direction with respect to arrow 34 and arrow 36. Although tissue expander 2 shown in FIG. 1A shows what shape tissue expander 2 is contemplated to attain when the fluid is disposed in outer shell 2, FIG. 2 shows what shape tissue expander 2 is contemplated to attain when the fluid is not disposed in outer shell 4 or when a small volume is present in outer shell 4.

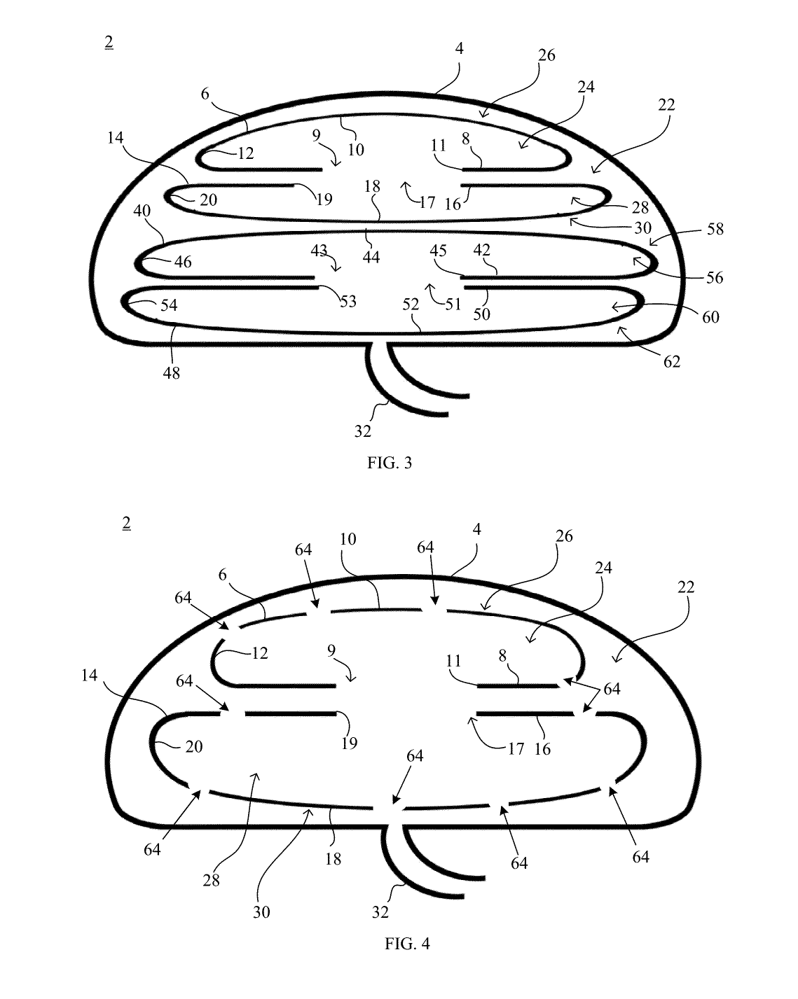

According to an embodiment, any number of open obloid shells (e.g., 6, 14, 40, 48, and the like) can be disposed in outer shell 4. In a particular embodiment, as shown in FIG. 3, first open obloid shell 6, second open obloid shell 14, third open obloid shell 40, and fourth open obloid shell 48 are disposed in outer shell 4 of tissue expander 2. Here, tissue expander 2 includes a first pair of obloid shells, the first pair of obloid shells being first open obloid shell 6 and second open obloid shell 14. Tissue expander 2 further includes a second pair of obloid shells disposed in outer shell 4, the second pair of obloid shells including third open obloid shell 40 and fourth open obloid shell 48. Third open obloid shell 40 includes third annular face 42, third opening 43 disposed in third annular face 42 and bounded by third rim 45, third outward face 44 disposed opposing third annular face 42, and third annular sidewall 46 interposed between third annular face 42 and third outward face 44 to connect third outward face 44 to third annular face 42. Fourth open obloid shell 48 includes fourth annular face 50, fourth opening 51 disposed and fourth annular face 50 and bounded by fourth rim 53, fourth outward face 52 disposed opposing fourth annular face 50, and fourth annular sidewall 54 interposed between fourth annular face 50 and fourth outward face 52 to connect fourth outward face 52 to fourth annular face 50, wherein third open obloid shell 40 and fourth open obloid shell 48 are stackingly arranged in outer shell 4 such that third annular face 42 opposes fourth annular face 50, such that the first pair of obloid shells (6, 14) and the second pair of obloid shells (40, 48) are stackingly disposed in outer shell 4 wherein second outward face 18 opposes third outward face 44.

In some embodiments, as shown in FIG. 4, through hole 64 is disposed in open obloid shell (e.g., 6, 14, 40, or 48). Through hole 64 provides fluid communication between interior 22 of outer shell 4 and interior (e.g., 24, 28, 56, or 60) of open obloid shell (e.g., 6, 14, 40, or 48).

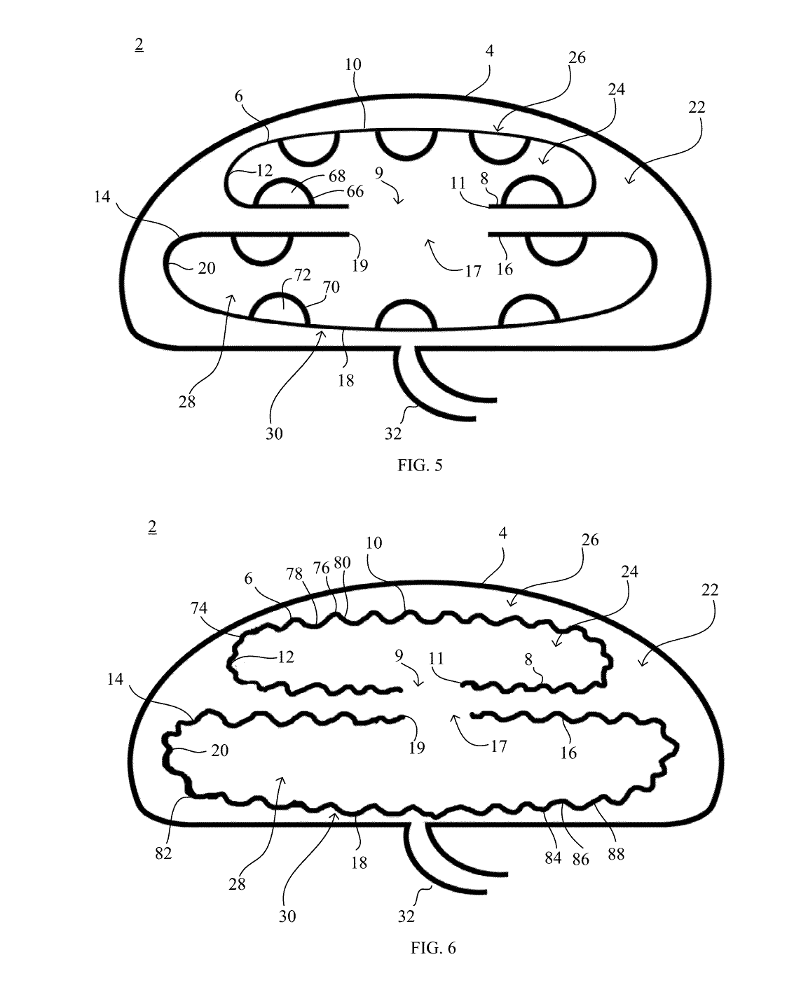

According to an embodiment, as shown in FIG. 5, tissue expander 2 includes first bubble 66 disposed on first open obloid shell 6. First bubble 66 can be disposed on interior 24 of first open obloid shell 6, exterior 26 of first open obloid shell 6, or a combination thereof. Tissue expander 2 also can include second bubble 70 disposed on second open obloid shell 14. Second bubble 70 can be disposed on interior 28 of second open obloid shell 14, exterior 30 of second open obloid shell 14, or a combination thereof. First bubble 66 or second bubble 70 respectively can include interior 68 or interior 72, which can be hollow, porous, or solid. When hollow or porous, interior 68 and interior 72 independently can be in fluid communication with interior 22 or can be isolated and not in fluid communication with interior 22. In some embodiments, when interior 68 or interior 72 is hollow or porous, interior 68 or interior 72 can be fluid-filled with a fluid that is different or the same as a fluid present in interior 24 or interior 28.

In an embodiment, as shown in FIG. 6, tissue expander 2 includes first annular face 8, first outward face 10, first annular sidewall 12, or a combination thereof that independently includes sinusoidally contoured surface 74. Moreover, second annular face 16, second outward face 18, second annular sidewall 20, or a combination thereof independently includes sinusoidally contoured surface 74. Here, sinusoidally contoured surface 74 includes projection 76, trough 78 disposed between projections 76, and smooth transition 80 that interconnects projection 76 to trough 78. Tissue expander 2 is a three-dimensional article such that sinusoidally contoured surface 74 extends in three dimensions.

According to an embodiment, first annular face 8, first outward face 10, first annular sidewall 12, or a combination thereof independently includes a smooth contoured surface (e.g., as shown in FIG. 1A). In some embodiments, second annular face 16, second outward face 18, second annular sidewall 20, or a combination thereof independently includes a smooth contoured surface (e.g., as shown in FIG. 1A).

In an embodiment, first outward face 10, second outward face 18, or a combination thereof includes an asymmetrically curved surface, wherein an entire portion of such face (10 or 18) is not symmetric. In a certain embodiment, first outward face 10, second outward face 18, or a combination thereof includes a substantially planar surface. In a particular embodiment, first outward face 10, second outward face 18, or a combination thereof includes a concave surface with respect to interior 24 or interior 28 of such faces. In a certain embodiment, first outward face 10, second outward face 18, or a combination thereof includes a convex surface with respect to interior 24 or interior 28 of such faces.

According to an embodiment, outer container 4, first open obloid shell 6, second open obloid shell 14 independently includes a flexible polymer that is present in an FDA-approved tissue expander. In some embodiments, the flexible polymer is silicone.

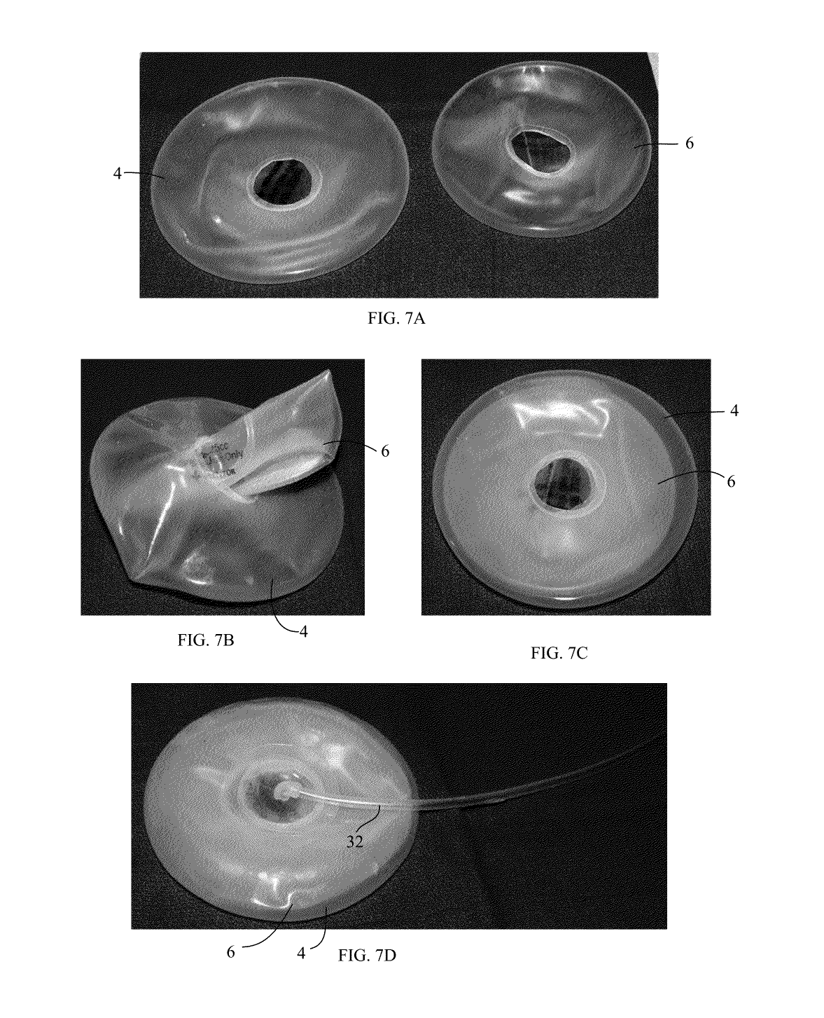

With reference to FIGS. 7A, 7B, 7C, and 7D, in an embodiment, a process for making tissue expander 2 includes providing outer shell 4, providing first open obloid shell 6 (FIG. 7A), disposing first open obloid shell 6 in outer shell 4 (FIG. 7B), attaching a patch to the posterior surface of outer shell 4 on which is disposed filling tube 32 to form tissue expander 2 (FIG. 7D). FIG. 7C shows an anterior view of expander 2 before attachment of filling tube 32.

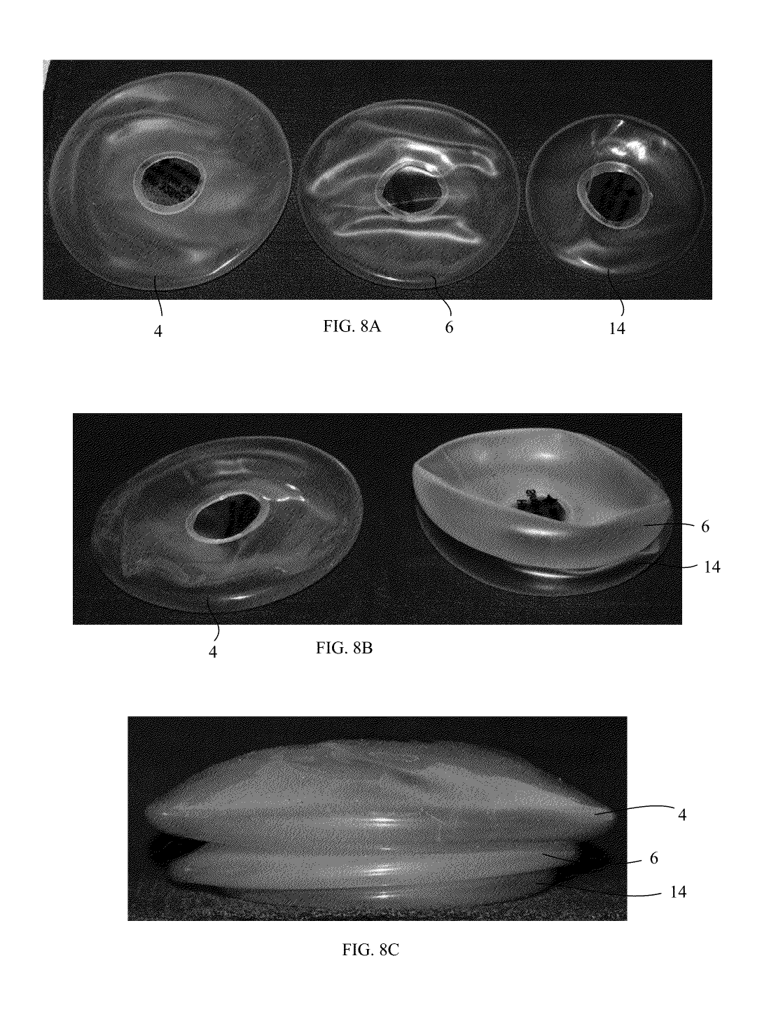

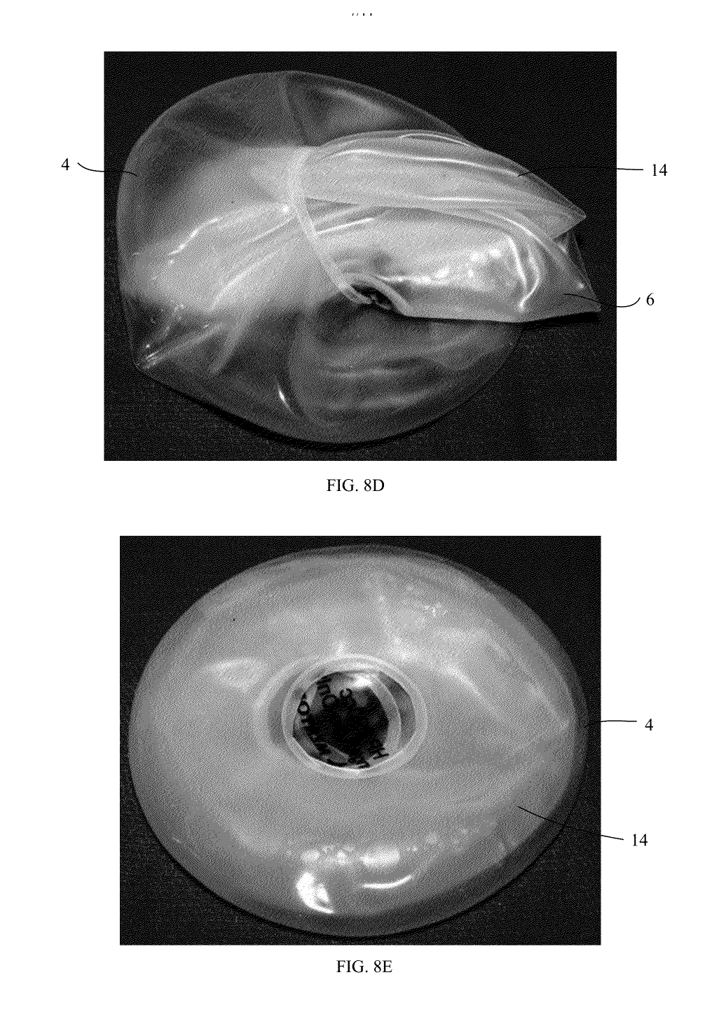

With reference to FIGS. 8A, 8B, and 8C, in an embodiment, a process for making tissue expander 2 includes providing outer shell 4, providing first open obloid shell 6, and providing second open obloid shell 14 (FIG. 8A); stacking first open obloid shell 6 on second open obloid shell 14 (FIG. 8B); and disposing first open obloid shell 6 and second open obloid shell 14 in outer shell 4 (FIG. 8D) to form tissue expander 2 (FIG. E). A patch can be attached to the posterior surface of outer shell 4 on which can be disposed filling tube 32. FIG. 8C shows a side view of outer shell 4, first open obloid shell 6, and second open obloid shell 14 in a stacked configuration.

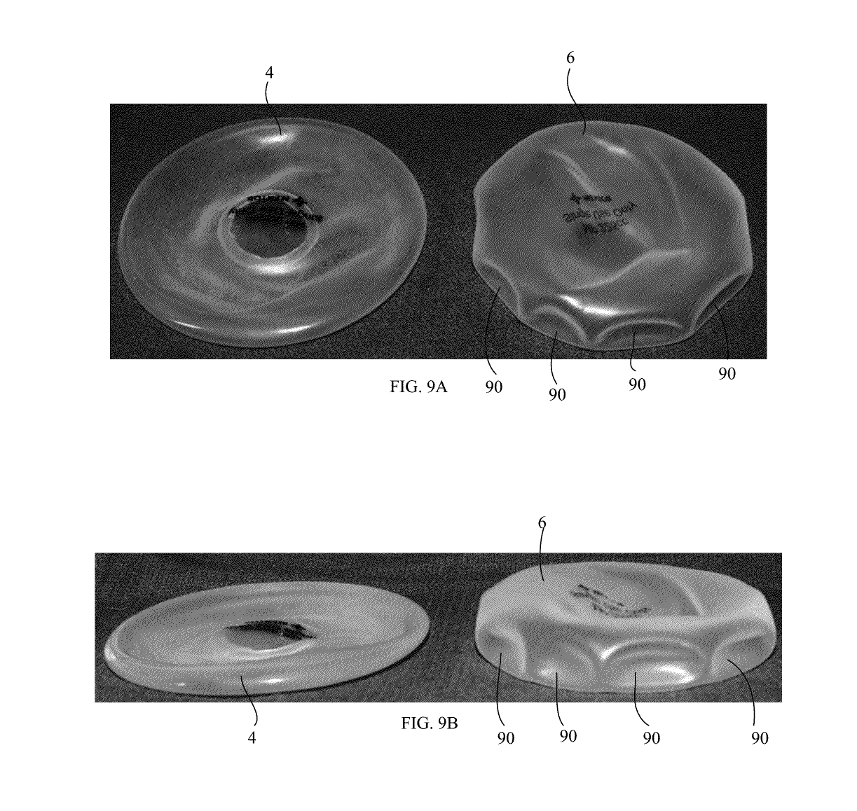

In an embodiment, tissue expander 2 can include outer shell 4 and first open obloid shell 6 that includes a plurality of dimples 90 disposed along first annular sidewall 12 as shown in FIG. 9A (a top view) and FIG. 9B (side view), wherein first open obloid shell 6 would be disposed in outer shell 4. Here, tissue expander 2 also can include a second open obloid shell 6 that independently includes a plurality of dimples 90.

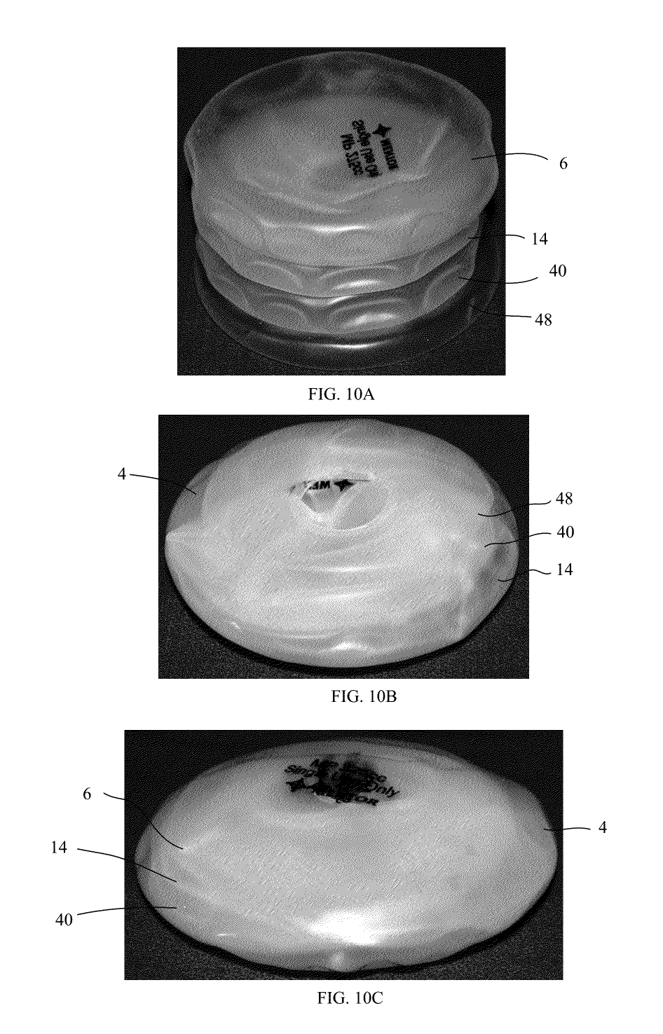

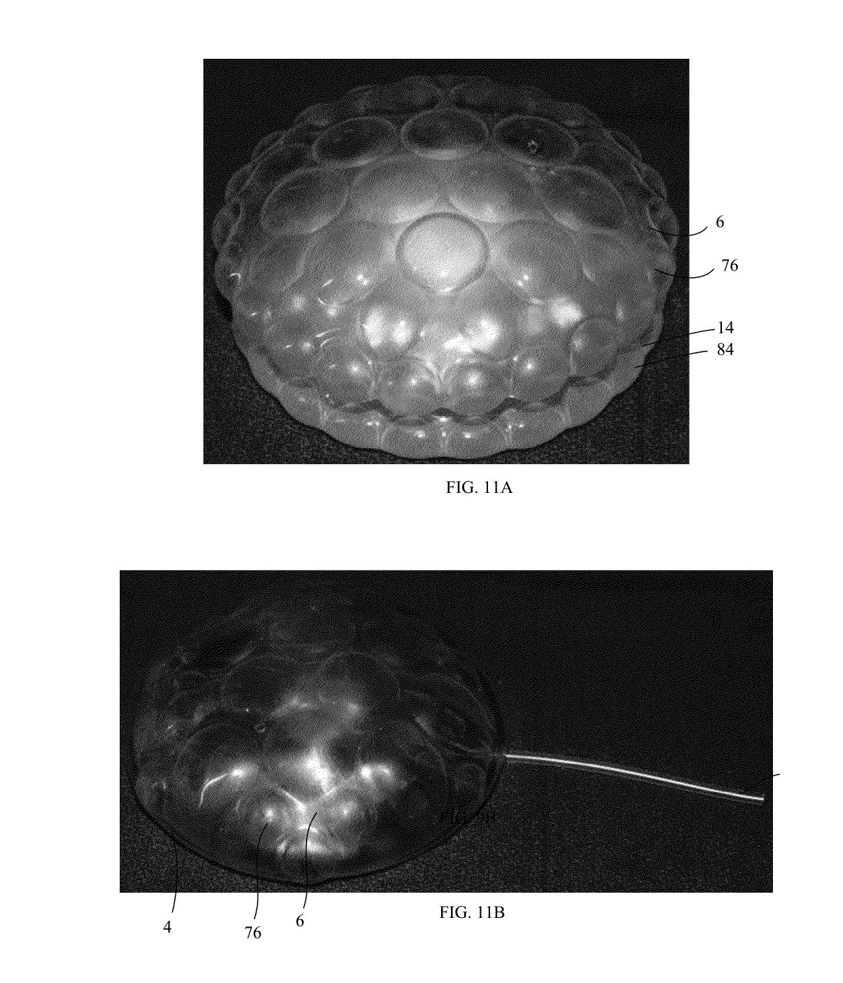

With reference to FIGS. 10A, 10B, and 10C, in an embodiment, a process for making tissue expander 2 includes providing first open obloid shell 6 has dimples 90 that are a result of turned first open obloid shell 6 inside out, second open obloid shell 14 having dimples 90, third open obloid shell 40 having dimples 90, and fourth open obloid shell 48 having dimples 90 in a stacked configuration (FIG. 10A); and disposing the stacked obloid shells (6, 14, 40, 48) in outer shell 4 (FIG. 10B). FIG. 10C shows an anterior view of tissue expander 2.

With reference to FIGS. 11A and 11C, in an embodiment, a process for making tissue expander 2 includes providing first open obloid shell 6 having sinusoidal surface 74 and includes projections 84, providing second open obloid shell 14 having sinusoidal surface 74 and includes projections 84, stacking first open obloid shell 6 on second open obloid shell 14 (FIG. 11A), and disposing the stacked obloid shells (6, 14) in outer shell 4 (FIG. 11B) to form tissue expander 2.

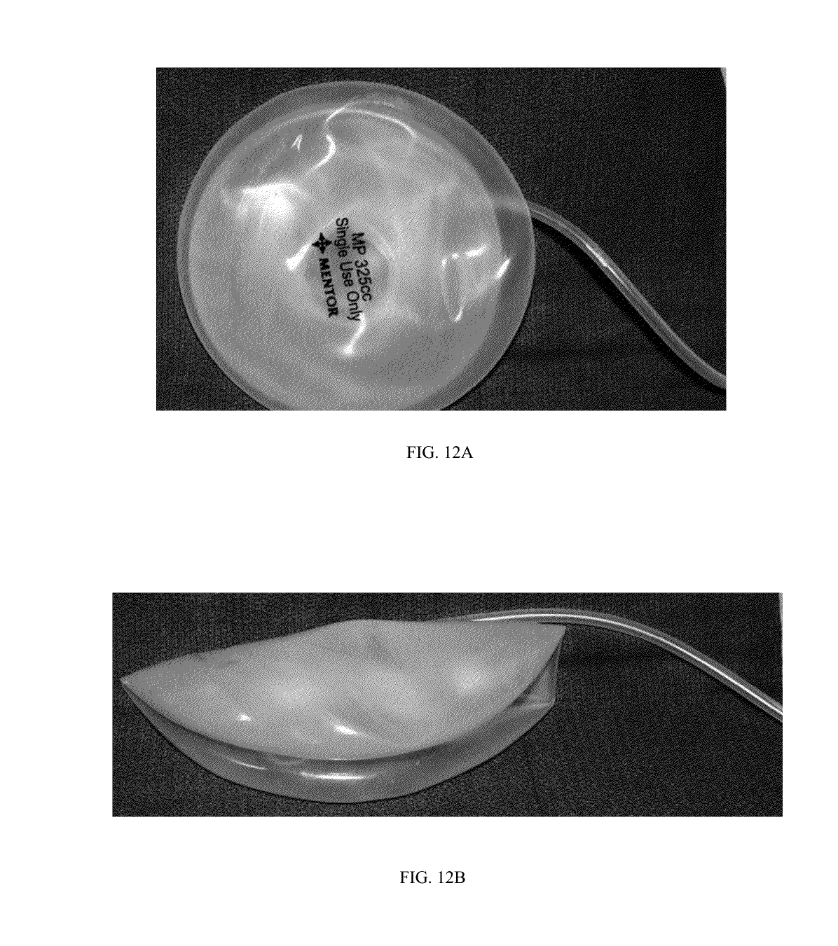

Tissue expander 2 can be applied as a tissue expander, expander implant, breast implant, or other implant placed within a human body. Further, tissue expander 2 that includes first open obloid shell 6 and second open obloid shell 14 disposed in outer shell 4 has internal structural support provided by first open obloid shell 6 and second open obloid shell 14 such that tissue expander 2 exhibits less rippling or scalloping than an outer shell that does have an open obloid shell. Further, tissue expander 2 does not fold (FIG. 12A) when evacuated and placed in a vertical orientation as compared with an outer shell that does not include an open obloid shell (FIG. 12B).

While one or more embodiments have been shown and described, modifications and substitutions may be made thereto without departing from the spirit and scope of the invention. Accordingly, it is to be understood that the present invention has been described by way of illustrations and not limitation. Embodiments herein can be used independently or can be combined.

Reference throughout this specification to “one embodiment,” “particular embodiment,” “certain embodiment,” “an embodiment,” or the like means that a particular feature, structure, or characteristic described in connection with the embodiment is included in at least one embodiment. Thus, appearances of these phrases (e.g., “in one embodiment” or “in an embodiment”) throughout this specification are not necessarily all referring to the same embodiment, but may. Furthermore, particular features, structures, or characteristics may be combined in any suitable manner, as would be apparent to one of ordinary skill in the art from this disclosure, in one or more embodiments.

All ranges disclosed herein are inclusive of the endpoints, and the endpoints are independently combinable with each other. The ranges are continuous and thus contain every value and subset thereof in the range. Unless otherwise stated or contextually inapplicable, all percentages, when expressing a quantity, are weight percentages. The suffix “(s)” as used herein is intended to include both the singular and the plural of the term that it modifies, thereby including at least one of that term (e.g., the colorant(s) includes at least one colorants). “Optional” or “optionally” means that the subsequently described event or circumstance can or cannot occur, and that the description includes instances where the event occurs and instances where it does not. As used herein, “combination” is inclusive of blends, mixtures, alloys, reaction products, and the like.

As used herein, “a combination thereof” refers to a combination comprising at least one of the named constituents, components, compounds, or elements, optionally together with one or more of the same class of constituents, components, compounds, or elements.

All references are incorporated herein by reference.

The use of the terms “a” and “an” and “the” and similar referents in the context of describing the invention (especially in the context of the following claims) are to be construed to cover both the singular and the plural, unless otherwise indicated herein or clearly contradicted by context. “Or” means “and/or.” Further, the conjunction “or” is used to link objects of a list or alternatives and is not disjunctive; rather the elements can be used separately or can be combined together under appropriate circumstances. It should further be noted that the terms “first,” “second,” “primary,” “secondary,” and the like herein do not denote any order, quantity, or importance, but rather are used to distinguish one element from another. The modifier “about” used in connection with a quantity is inclusive of the stated value and has the meaning dictated by the context (e.g., it includes the degree of error associated with measurement of the particular quantity).

What is claimed is:

1. A tissue expander comprising:
an outer shell;
a first open obloid shell disposed in the outer shell, the first open obloid shell comprising:
a first annular face;
a first outward face disposed opposing to the first annular face; and
a first annular sidewall interposed between the first annular face and the first outward face to connect the first outward face to the first annular face; and
a second open obloid shell disposed in the outer shell, the second open obloid shell comprising:
a second annular face;
a second outward face disposed opposing to the second annular face; and
a second annular sidewall interposed between the second annular face and
the second outward face to connect the second outward face to the second annular face,
wherein the first open obloid shell and the second open obloid shell are stackingly arranged in the outer shell such that the first annular face opposes the second annular face.

2. The tissue expander of claim 1, further comprising a first bubble disposed on the first open obloid shell.

3. The tissue expander of claim 2, wherein the first bubble is disposed on an interior of the first open obloid shell, and the first bubble is in fluid communication with an interior of the outer shell.

4. The tissue expander of claim 2, wherein the first bubble is disposed on an exterior of the first open obloid shell.

5. The tissue expander of claim 2, further comprising a second bubble disposed on the second open obloid shell.

6. The tissue expander of claim 5, wherein the second bubble is disposed on an interior of the second open obloid shell.

7. The tissue expander of claim 5, wherein the second bubble is disposed on an exterior of the second open obloid shell.

8. The tissue expander of claim 1, further comprising a filling tube disposed on the outer shell and comprising an inner flow path that is in fluid communication with an interior of the outer shell.

9. The tissue expander of claim 8, wherein the filling tube is in fluid communication with the first open obloid shell and the second open obloid shell.

10. The tissue expander of claim 1, further comprising a first pair of obloid shells, the first pair of obloid shells comprises the first open obloid shell and the second open obloid shell.

11. The tissue expander of claim 10, further comprising a second pair of obloid shells disposed in the outer shell, the second pair of obloid shells comprising:
a third open obloid shell comprising:
a third annular face;
a third outward face disposed opposing to the third annular face; and
a third annular sidewall interposed between the third annular face
and the third outward face to connect the third outward face to the third annular face; and
a fourth open obloid shell comprising:
a fourth annular face;
a fourth outward face disposed opposing to the fourth annular face; and
a fourth annular sidewall interposed between the fourth annular face and the fourth outward face to connect the fourth outward face to the fourth annular face,
wherein the third open obloid shell and the fourth open obloid shell are stackingly arranged in the outer shell such that the third annular face opposes the fourth annular face,
such that the first pair of obloid shells and a second pair of obloid shells are stackingly disposed in the outer shell wherein the second outward face opposes the third outward face.

12. The tissue expander of claim 1, wherein the first annular face, the first outward face, the first annular sidewall, or a combination comprising at least one of foregoing comprises a sinusoidally contoured surface.

13. The tissue expander of claim 1, wherein the second annular face, the second outward face, the second annular sidewall, or a combination comprising at least one of foregoing comprises a sinusoidally contoured surface.

14. The tissue expander of claim 1, wherein the first annular face, the first outward face, the first annular sidewall, or a combination comprising at least one of foregoing comprises a smooth contoured surface.

15. The tissue expander of claim 1, wherein the second annular face, the second outward face, the second annular sidewall, or a combination comprising at least one of foregoing comprises a smooth contoured surface.

16. The tissue expander of claim 1, wherein the first outward face, the second outward face, or combination comprising at least one of the foregoing faces comprises an asymmetrically curved surface.

17. The tissue expander of claim 1, wherein the first outward face, the second outward face, or combination comprising at least one of the foregoing faces comprises a substantially planar surface.

18. The tissue expander of claim 1, wherein the first outward face, the second outward face, or combination comprising at least one of the foregoing faces comprises a concave surface.

19. The tissue expander of claim 1, wherein the first outward face, the second outward face, or combination comprising at least one of the foregoing faces comprises a convex surface.

20. The tissue expander of claim 1, wherein the outer container, first open obloid shell, and the second open obloid shell independently further comprise a flexible polymer that is present in an FDA-approved tissue expander.

21. The tissue expander of claim 1, wherein outer shell comprises a shape in transverse cross-section that is a spherical shape or an oval shape, the oval shape approximating an anatomical shape for healthy human breast tissue.
